(19) United States
(12) Patent Application Publication
(10) Pub. No.: US 2015/0021408 A1
(43) Pub. Date: Jan. 22, 2015

(54) CUSTOMIZABLE SCENT DISPENSER

(71) Applicant: Samuel David Matthews, Hendersonville, NC (US)

(72) Inventor: Samuel David Matthews, Hendersonville, NC (US)

(21) Appl. No.: 13/946,076

(22) Filed: Jul. 19, 2013

(51) Int. Cl. A61L 9/12

(52) U.S. Cl. CPC A61L 9/12
USPC 239/34

(57) ABSTRACT

A customizable scent dispenser is provided that includes an outer case, a scent emitting element, and an inner enclosure operable to support the scent emitting element within the outer case in a manner in which at least a portion of the scent emitting element is supported at a spacing from the outer case so as to not be in direct contact therewith. The inner enclosure has an aperture through which replenishment access to the scent emitting element can be had such that a scented composition can be imparted to the scent emitting element.

### BACKGROUND OF THE INVENTION

This present invention relates to a customizable s dispenser having an outer case, an inner enclosure, and a wick/fragrance source for disseminating agreeable odors such as perfumes, aromas, and fragrances.

The present invention provides a wick/fragrance source that is refreshable in the manner that the wick/fragrance source is renewable in place (e.g., while supported within the outer case), renewable via temporary extraction, replenishment and repositioning again within the outer case, or replaceable with a fresh unit of the wick/fragrance source. This versatility adds a high degree of consumer convenience and freedom to personally select a desired fragrance. In this manner, the inventive customized scent dispenser of the present invention provides an aesthetic and economical alternative to known sachet or fragrance holders and dispensers which utilize non-replaceable non-renewable, scent packets permanently fabricated within the sack or which provide a relatively unattractive means for receiving a scent element.

Sachet or fragrance holders carrying various perfumes and enlivening the olfactory senses have long been recognized as a means for supplementing and enhancing pleasant odors with additional agreeable odors. In the art of sachet, for example, there are a variety of arrangements for adding a fragrance by the placement of a scent carrying packet within a sack and the entire sack and content remnants are typically thrown away after the fragrance has substantially diminished. A sachet may utilize a package of scent carrying material such as, for example, rose petals or dried herbs, which emit a characteristic smell.

A particular challenge with sachets is that the fragrance dissipates very rapidly. In fact, in some cases, the sachets lose their fragrance almost entirely between manufacture and purchase, which results in either an unhappy customer or a lost sale. Another drawback for the consumer is that the sachet is often provided with a scent package permanently sewn in. Thus, a consumer seeking a greater range of fragrance options, or seeking to obtain full economic value from a sachet purchase is nonetheless left with a purchase choice which is necessarily of a permanent nature. Moreover, the choice is often relatively limited in that every fragrance package may not be available in each decorator design.

A wide variety of constructions have been utilized in the prior art for providing fragrance sacks. Frequently, however, such sacks have featured a decorative cover for powdered scenting material and carried the scent producing material in an air permeable package. The package would then be pinned to clothing or placed in close proximity to that which a characteristic smell is to be imparted. Alternatively, the scent emitting powder has been pieced within perforated and wire lined lids of a can-type container. A common characteristic of these existing products is the need to replace the whole scenting structure or the need to otherwise undertake a careful freshening of a pad while it remains in place in a carrier. For example one construction of a sachet has an extending ribbon end closure providing access to an unstuffed cavity which receives a charge of perfumed powder. This design, however, requires the unstuffing of the entire article to enfold a quantity of scented powder into a cotton mass to provide a fresh charge of powder. Similarly, another design uses an adhesive adornment to carry a scented pellet in an open pocket cut into the front face of a two layer stitched construction. However, this design provides no retaining means for the scenting element, and in addition, the scent saturated pellet resides on the backside of the decorative facing material thus subjecting the facing to straining. Thus, it can be easily understood the range of usefulness of these existing product has been somewhat limited.

These limitations of disassembly for replacement replacing a powder charge, simple placement in an open pocket, and low protection of the carrier from stain or damage by the scent element are inherent in the existing sachet or fragrance emitting products. Accordingly, while such products have achieved at least a degree of recognition in the area of fragrance enhancement, there is room for significant improvement.

### SUMMARY OF THE INVENTION

It is one object of the invention to provide a new and different scent emitting product which will obviate or minimize difficulties of the type previously described.

It is a specific object of the invention to provide a novel fragrance scent emitting product for providing a means for scenting garments in a closet or dresser drawer.

It is a specific object of the invention to provide a novel self contained fragrance scent emitting product for providing means to scent foods stored in proximity to the scent emitting product.

It is another object of the invention to provide a novel fragrance scent emitting product having provision to carry replaceable scenting elements which ma be advantageously employed in homes and residences during holiday seasons to give an appealing fragrance to the residential surroundings.

It is yet another object of the invention to provide a novel fragrance scent emitting product for carrying a replaceable scent element in an open pocket which can be placed in proximity to a heat source to disperse a desirable fragrance throughout a living area by the convective heat flow of the air

It is a further object of the invention to provide a novel attractively adorned fragrance scent emitting product having a releasable, replaceable and retainable scent element retained in a decorative outer case.

It is yet a further object of the invention to provide a new and different scent emitting product for placement in a residential area, an office area, or other occupied space and which has a renewable scent element that may be custom scented for advantageous blending with the prevailing fragrance character of the occupied space.

It is still another object of the invention to provide a novel free formed fragrance scent emitting product made into the desirable shapes of Christmas ornaments or other such ornaments for hanging on a Christmas tree; such a scent emitting product having refreshable scenting elements useable season after season.

### BRIEF DESCRIPTION OF THE DRAWINGS

Other objects and advantages of the present invention will become apparent from the following detailed description of an embodiment thereof taken in conjunction with the accompanying drawings wherein:

FIG. 1 is a side elevational schematic view of one embodiment of the customizable scent dispenser of the present invention;

FIG. 2 is a front perspective view of the customizable scent dispenser of the present invention shown in FIG. 1 in its deployed condition;

FIG. 3 is an enlarged perspective view of the scent emitting element of customizable scent dispenser of the present invention shown in FIGS. 1 and 2;

FIG. 4 is a front plan view of one variation of the customizable scent dispenser of the present invention;

FIG. 5 is a rear perspective view of another variation of the customizable scent dispenser of the present invention;

FIG. 6 is a front perspective view of a configuration of the customizable seen dispenser of the present invention as a greeting card kit;

FIG. 7 is a perspective view of a variation of the one embodiment of the customizable scent dispenser of the present invention shown in FIGS. 1-3; and

FIG. 8 is a perspective view of a further variation of the one embodiment of the customizable scent dispenser of the present invention shown in FIGS. 1-3.

### DETAILED DESCRIPTION OF AN EMBODIMENT OF THE INVENTION

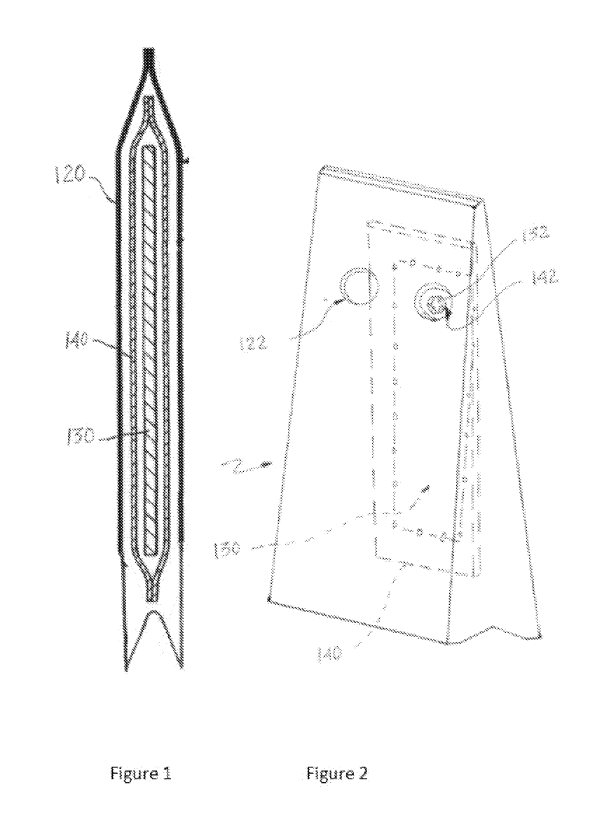

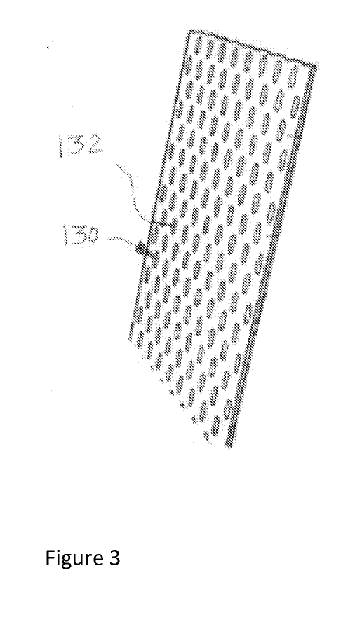

One embodiment of the customizable scent dispenser of the present invention is shown in FIGS. 1-3 and is generally designated as the customizable scent dispenser 110, The customizable scent dispenser 110 includes an outer case 120, a scent emitting element 130, and an inner enclosure 140 operable to support the scent emitting element 130 within the outer case 120 in a manner in which at least a portion of the scent emitting element 130 is supported at a spacing from the outer case 120 so as to not be in direct contact therewith. The outer case 120 is configured as a foldable apex structure deployable between a collapsed disposition, as shown in FIG. 1, and a deployed disposition shown in FIG. 2, in which the outer case has been fully folded out. The scent emitting element 130, which is shown in FIG. 2 in broken dash-dot lines, is formed of an absorbent paper fiber material and is infused with a scented composition. As seen in FIG. 3, which is an enlarged perspective view of the scent emitting element 130, the scent emitting element has a plurality of depressions 132 whereat the absorbent paper fiber material is formed in more closely packed layers than the surrounding area. The scent emitting element 130 is enclosed within the inner enclosure 140 which is in the form of a pouch having an aperture 142 through which replenishment access to the scent emitting element 130 can be had such that a scented composition can be imparted to the scent emitting element 130. The inner enclosure 140 is shown in broken lines in FIG. 2. For example, fresh oil or another fragrance producing composition can be delivered onto the customizable scent dispenser 110 through the aperture 142 in the inner enclosure 140 and this replenishment operation can be performed without the need to withdraw the scent emitting element 130 from the inner enclosure 140.

A portion of the outer case 120 delimits a refresh access path via which access to the inner enclosure 140 can be had for the purpose of selectively (a) replenishing the scent emitting element 1130 with a scented composition, with access to the scent emitting element 130 occurring through the aperture 142 of the inner enclosure 140; (b). removing the inner enclosure 140 for replenishing the scent emitting element 130 with a scent providing composition while the inner enclosure 140 and the therein retained scent emitting element 130 are removed from the outer case 120, or (c): removing the inner enclosure 140 with the therein retained scent emitting element 130 and substituting a new inner enclosure with its own respective scent emitting element retained therein. The refresh-access path may be, for example, an opening 122 formed in the outer case or any one of a plurality of openings 122 formed in the outer case. FIG. 2 shows that the outer case 120 has a pair of openings 122 on each longitudinal planar side and it can be seen that access to the aperture 142 of the inner enclosure 140 can be had via one of these openings 122 in the outer case 120 and, in turn, access to the scent emitting element 130 can be had via the aperture 142 of the inner enclosure 140.

The outer case 120 can be formed of a paper sheet that maintains its integrity and shape even if subjected to limited moisture contact and which may be comprised of a substantially planar cellulosic substrate, such as a sheet of paper, where the substrate is coated on at least one side with a composition that, thereby, provides a durable weatherproofing coating layer. The composition comprises a copolymeric resin derived from styrenic and acrylic monomers, as well as a wax, such as paraffin wax; a filler to provide block resistance, such as barium sulfate; a filler, such as calcium carbonate, to provide tooth to the surface of the sheet; and a pigment, if desired, such as titanium dioxide to provide a white color to the sheet. The composition may comprise an emulsified styrenic acrylic copolymer or mixture of copolymers, an emulsified paraffin wax, barium sulfate as a filler to provide block resistance, calcium carbonate as a filler to provide a capability to imprint instructions and/or other written matter on the sheet, and titanium dioxide as a pigment to provide a white color to the sheet. The wax may be paraffin wax, a polypropylene-wax mixture, a polyethylene-wax mixture, carnauba wax, microcrystalline wax, montan wax, a Fisher-Tropsch wax, beeswax, or a mixture thereof.

The material the inner enclosure 140 is selected with a view toward an efficient transfer of desirable fragrances or scents through the material and, depending on the intended use, may be any suitable material such as cotton, silk, nylon, wool or other woven or knitted fabric. Alternatively, the inner enclosure 140 can be formed of a synthetic material such as a plastic sheet with holes therein or a vapor permeable plastic sheet. The scent emitting element 130 may be formed from an absorbent material such as balsam, cotton, fibrous paper or any other suitable absorbing material and may be configured to absorb a volatile fluid such as a liquid fragrance essence, essential oils, or any of a class of volatile oils possessing the desirable odor properties.

The outer case 120 may be configured as a simple geometric construction such as an envelope or may be formed as a small box structure or may be configured as a complex geometric construction such as a multi fold or multi creased construction to mimic a three-dimensional (3-D) media character, a pleasing scene, or a corporate logo. The outer case 120 preferably include additional openings beyond the opening 122 and these additional openings will facilitate the escape of the scent vapors emitted by the scent emitting element 130 so that these vapors can eventually waft out to scent the desired environment. The additional openings and the opening 122 are preferably positioned so that it does not impair the visual attractiveness of the outer case 120.

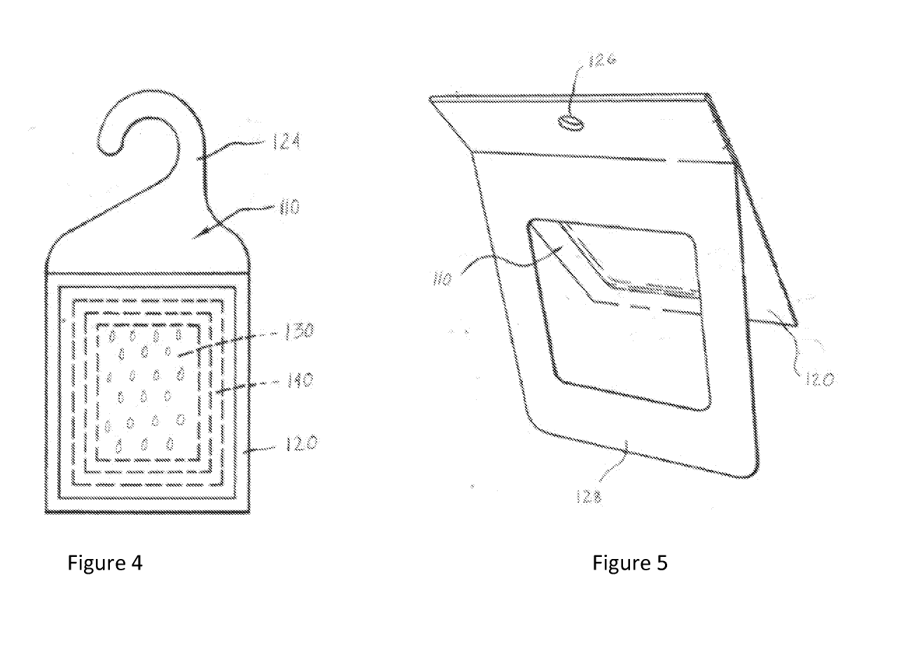

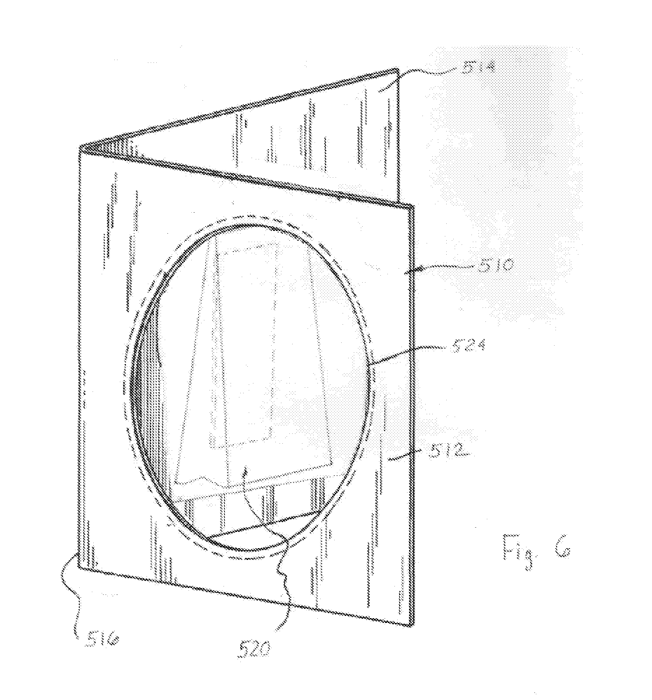

The outer case 120 may contain a color coordinated securing or hanging mechanism such as a stretchy elastic decorative cord or similar such attachment. As seen in FIG. 4, the outer case 120 may also have a preformed hook 124 to permit the outer case 120 to be hung or secured in a specific area. As seen in FIG. 5, the outer case 120 may be provided with a hook hang aperture 126 for hanging the customized scent dispenser from a string or the like. Alternatively, as also seen in FIG. 5, a lean-back frame 128 may be provided that is hingedly connected to the outer as 120 so that the customized scent dispenser can be self-standing on a table surface or a countertop.

It is thus submitted that the customized scent dispenser of the present invention can offer a very unique and simple scent delivery system that, in one aspect thereof, is basically a pouch within a pouch configuration consisting of only two components forming the inner pouch system (an inert inner enclosure and wick), and a single component forming the outer pouch system (an inert outer case), whereupon these three components in total provide a pouch within a pouch system. Some configurations of the inventive customized scent dispenser can offer a very inexpensive, reliable, economical, and reusable scent delivery system which is easy to manufacture, ship, and be marketed to consumers. If desired, the inventive customized scent dispenser in the form of a reusable scent delivery system can have a very long life and this is an improvement over the numerous “one trip use” air fresheners that are marketed today.

Moreover, the inventive customized scent dispenser can be configured as a scent delivery system that is so compact it is small enough to be used inside greeting cards or in a small area such as school lockers, car and kit bag and equally also able to be used in larger areas such as different rooms in a home, offices or shop and provide a healthy, welcoming and relaxed environment in each. In further connection with this capability to provide compactness and portability, one configuration of the inventive customized scent dispenser can be as a scent delivery system that is very light weight and can be hung from a clothes hanger in a closet or even on a Christmas tree branch with no difficulty or mailed in an envelope with little additional postage charge.

With respect to the environmentally beneficial aspects of the inventive customized scent dispenser, the inventive customized scent dispenser can be configured with a significant degree of natural, recyclable, renewable, and/or sustainable materials. Furthermore, in many applications, the inventive customized scent dispenser does not require flame or fire, a liquid reservoir, electric energy, one trip non recyclable plastic containers, or bulky solid fragrances to create a healthy, welcoming and relaxed environment.

The inventive customized scent dispenser makes it possible for a scent delivery system to be made available to the customer in a natural and unscented form unlike every other air freshener system on the market today. The inventive customized scent dispenser offers the consumer the ability to completely customize the scent emitted by the inventive customized scent dispenser—dependent only on their imagination and the availability of scented liquid volatile ingredients. The inventive customized scent dispenser can easily be designed to be a multiple reusable system that allows the consumer to recharge the wick system time after time and without actually touching the wick with their hands during the recharging operation. This non-contact recharging procedure can be achieved, for example, via dripping or dropping a new flavor charge through the aperture(s) in the inner enclosure and directly onto an absorbing wick acting as the scent emitting element. Moreover, this non-contact recharging procedure is highly beneficial if the new flavor charge being administered to the wick is an insecticide or pesticide material where it would be advantageous not to have to touch the wick directly. If a completely new scent or flavor is desired, the absorbent wick can be easily removed and discarded or re used and a new unscented absorbent pad can be introduced to the inner enclosure.

With regard to the beneficial presentation and display aspects of the inventive customized scent dispenser, the inventive customized scent dispenser can include an attractive themed or custom printed outer case. Further customization features such as allowing customers to use their own custom scented fragrance take advantage of the fact that new and exotic oil combinations are constantly being formulated and offered for sale, particularly on the Internet, and so customers can update the scent offerings while reusing the outer case and the inner enclosure through numerous wick recharging procedures. The inventive customized scent dispenser can thus be a suitable vehicle for dispensing scent from oil formulators that not only offer pleasant smells but also purport to offer curative properties—i.e., the popular alternative healthy medicinal benefits that aromatherapy is purported to have on the brain and, particularly, on mood, emotion and health.

With the current new scent delivery system being offered a consumer can try these new oil formulations and very quickly and cheaply and verify the touted performance or fragrance claims. All current scented air freshening solutions come pre-formulated from the factory—these can be very pleasing or very not so much pleasing to the individual—while, in contrast, with the inventive customized scent dispenser, complete scent customization can be offered to the customer using their own creativity and ingredient concentration and combinations.

In accordance with an additional aspect of the present invention, the customized scent dispenser is configured as a greeting card. As seen in FIG. 6, a greeting card generally designated as the greeting card 510 has a front portion 512 and a rear portion 514 generally joined along a common edge 516. The common edge 516 avows the front portion 512 and the rear portion 514 to be folded upon each other (such as would be the position when inserted in an envelope for mailing) or permits the two portions 512 and 514 to be opened away from each other and positioned in a generally singular plane. The greeting card 510 has a removable scent customized scent dispenser 520 secured to the rear portion 514 by an adhesive material. The front portion 512 has an opening 524 therethrough to allow viewing of the removable scent customized scent dispenser 520 when the card 510 is in the folded closed position and/or to permit the removable scent customized scent dispenser 520 protrude partially therethrough so that the front portion 512 and the rear portion 514 can be folded upon each other. Thus, the greeting card 510 can be mailed to or otherwise presented to a recipient and the recipient can choose to display the greeting card while the removable scent customized scent dispenser 520 remains secured to the greeting card or to remove the removable scent customized scent dispenser 520 from the greeting card and then deploy the removable scent customized scent dispenser 520 in a selected location.

As an alternative to providing the customized scent dispenser 510 with a selected scent already applied to the scent emitting element 530, the customized scent dispenser 510 can be configured with a separate container such as, for example, a small cylindrical closed cap vial, that contains a dosage of a scent producing substance. This separate container can be removably secured to the greeting card. This provides an opportunity for a greeting card purchaser to send a nicely textured and visually pleasing card along with a meaningful text message and, additionally, to send the customized scent delivery system along with a personally chosen fragrance that can applied at the discretion of the greeting card recipient. The greeting card recipient may also choose to apply a different scent or fragrance of their own choosing. As the card would be retailed in an unscented form, this avoids an over powering and eye watering shopping experience when buying the card. Since the customized scent dispenser can be configured in such a compact and light design, the customized scent dispenser can be offered as an additional element of a premium greeting card. This allows greeting card retailers to offer an entirely new category of greeting card where, in addition to a pleasing visual and texture of the card along with a meaningful text message, a customizable scent delivery system can be purchased.

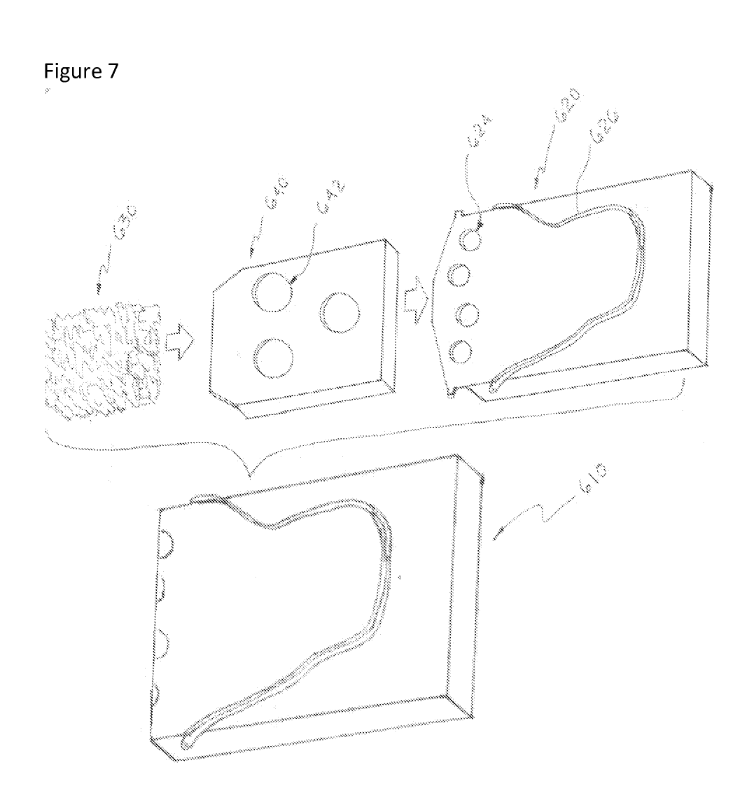

Reference is now had to FIG. 7, which is a perspective view of a variation of the one embodiment of the customizable scent dispenser of the present invention shown in FIGS. 1-3. This variation illustrates the simplicity of the construction of the customized scent disperser that can be achieved as well as it the economic use of materials that nonetheless reliably provide a product that can be repeatedly recharged with a scent or fragrance. As seen in FIG. 7, which shows an exploded view of the components of the customized scent dispenser, a customizable scent dispenser 610 includes an outer case 620, a scent emitting element 630, and an inner enclosure 640 operable to support the scent emitting element 630 within the outer case 620 in a manner in which at least a portion of the scent emitting element 630 is supported at a spacing from the outer case 620 so as to not be in direct contact therewith. The cuter case 20 is configured as a rectangular box having a relatively modest thickness and having a foldable flap at one end. The scent emitting element 630 is formed of an absorbent material and is infused with a scented composition and it can be seen that the scent emitting element 630 is a pad of absorbent cotton batting.

The scent emitting element 630 is enclosed within the inner enclosure 640 which is in the form of a rectangular pouch having a plurality of apertures 142 through which replenishment access to the scent emitting element 630 can be had such that a scented composition can be imparted to the scent emitting element 630. For example, fresh oil or another fragrance producing composition can be delivered onto the customizable scent dispenser 610 through the apertures 642 in the inner enclosure 640 and this replenishment operation can be performed without the need to withdraw the scent emitting element 630 from the inner enclosure 640. The inner enclosure 640 is sized in correspondence with the rectangular box-shaped outer case 620 so that the inner enclosure 640 (with the scent emitting element 630 seated inside it) can be slid into the cuter case 620 and retained therein by closure of the foldable flap of the outer case 620 and can be slid out of the outer case 620 for a scent recharging operation.

The outer case 20 has a plurality of openings 624 that permit scent vapors to exit the customized scent dispenser. The outer case 620 has a hang strap 626 that permits the customized scent dispenser to be conveniently suspended from, for example, a closet rail, a window sill, or on a hook. Thus, it can be understood that the customized scent dispenser 610 provides “a vented pouch within a vented pouch” configuration and this variation of the customized scent dispenser can be compactly configured, can be formed completely of biodegradable materials, and can operate as a decorative yet non-stain producing self-contained scent dispenser. It can also be seen that the customized scent dispenser 610 provides an attractive configuration for a car freshener—i.e. the customized scent dispenser 610 is capable of being scented with custom fragrance and used to provide a personal pleasing environment within a vehicle. A user can hang the customized scent dispenser 610 via its hang strap 626 from, for example, the mounting stem of an interior car mirror or from a sun visor

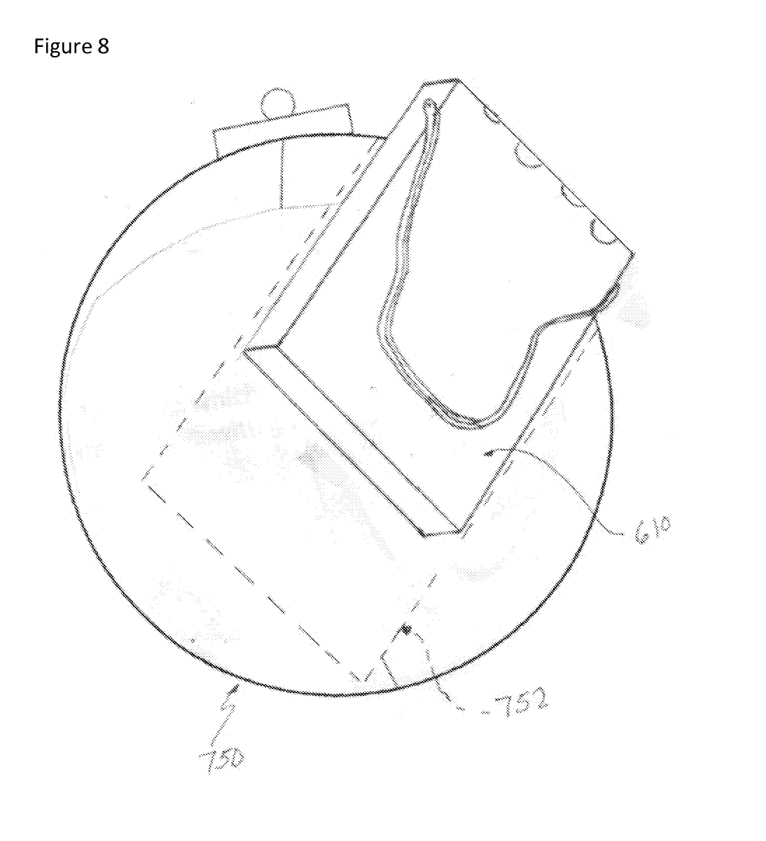

Reference is now had to FIG. 8, which is a perspective view or a further variation of the one embodiment of the customizable scent dispenser of the present invention shown in FIGS. 1- 3. This variation illustrates that the customized scent dispenser is easily combinable with other decorative or functional accessories and an example is provided here that the customized scent dispenser can be provided as part of a holiday decoration kit that includes a holiday tree ornament, such as, for example, a Christmas tree ornament. The customized scent dispenser 610 that has been described with respect to FIG. 7 can be accommodated within a holiday tree ornament 750 that is configured to be hung on a branch of a natural or artificial tree. The holiday tree ornament 750 includes an insert slot 752 sized in correspondence with the customized scent dispenser 610 such that the customized scent dispenser can be slid into the insert slot 752 and retained therein via, for example, a friction fit, and can be slid out of the insert slot 752 for a scent recharging operation.

The customizable scent dispenser of the present invention allows not only custom scent selection but also allows a user to select the rate at which a scent or fragrance is dispensed. For example, if a user prefers a mild fragrance, the user restricts the quantity of the scent providing substance—i.e., adds only a drop or so on the wick. On the other hand, if a user prefers a stronger aroma, the user can add several drops to charge the wick with a heavier dose of scent. Thus, the customized scent dispenser is fully customizable to personal preference.

In accordance with a further aspect of the present invention, the customized scent dispenser is configured as a system that can retain and dispense a scent or a fragrance, whereupon the scent or fragrance is selected for its impact or influence on insects, pests, and the like. The customized scent dispenser in this configuration may operate to dispense a scent or fragrance typically via a long term natural evaporation or vaporization of the retained scent ingredients. The customized scent dispenser may be configured to retain vaporizable pesticide ingredients and thus serve as a pesticide delivery system.

The customized scent dispenser in a configuration as a pesticide delivery system may be formed as a scent delivery pouch that comprises a water resistant envelope having good wet strength and an insert fitted into the water resistant envelope that is infused with scent or pesticide ingredients or otherwise retains these volatile ingredients. The volatile ingredient insert may be formed of absorbent cotton pads. The water resistant envelope may include ventilation holes for outward passage of the scent vapors or the material forming the envelope may be configured to provide the vapor passage at the desired rate.

One advantage of the scent delivery pouch is that the volatile ingredient insert and the water resistant envelope can be offered in a small portable size. Moreover, with appropriate selection of the scent or pesticide ingredients, and with appropriate choice of materials for the volatile ingredient insert and the water resistant envelope, the entire scent delivery pouch can be an all natural, fully biodegradable product. For example, the scent or pesticide ingredients can be any one of a range of natural scent or bug repelling ingredients, the volatile ingredient insert can be formed of absorbent cotton pads, and the water resistant envelope can be formed of a water resistant paper material, including a water resistant paper material having water repelling agents that volatize or evaporate when the paper material is burned or heated (i.e., during a discarded waste incineration process).

The water resistant envelope can be formed of a paper sheet that maintains its integrity and shape even if subjected to limited moisture contact and which may be comprised of a substantially planar cellulosic substrate, such as a sheet of paper, where the substrate is coated on at least one side with a composition that, thereby, provides a durable weatherproofing coating layer. As used herein, the term “Water resistant” refers to the ability of the water resistant envelope of the scent delivery pouch to resist wetting that is, the passage of water into the structural components of the water resistant envelope of the scent delivery pouch through capillary action. Wet strength refers to the tensile strength of the water resistant envelope of the scent delivery pouch when permeated with water, the strength being provided by interfiber bonds resistant to attack by water.

Since the water resistant envelope can fully prevent or significantly retard the passage therethrough of water or liquids, the volatile ingredient insert and its retained scent or pesticide ingredients can be damp or moist and there will be no risk that undesirable oil stains or seepage will mar a surface on which the scent delivery pouch is placed. Additionally, the water resistant envelope can have a long life durability and can thus handle multiple fresh replacements units of the volatile ingredient insert.

The exemplary shapes, dimensions and materials described herein are provided by way of example only. Products fabricated in different shapes or dimensions and comprised of different features and materials other than those discussed and illustrated herein also are contemplated as being within the scope of the present invention.

Although this invention has been disclosed and described in its preferred forms with a certain degree of particularity, it is to be realized that the optimum dimensional relationships for the parts of the invention, to include variations in size, materials, shape, form, function and manner of operation, assembly and use, are deemed readily apparent and obvious to one skilled in the art. Additionally, it is understood that the present disclosure of the preferred forms is only by way of example and that numerous changes in the details of operation and in the combination and arrangement of parts may be resorted to without departing from the spirit and scope of the invention as hereinafter claimed.

What is claimed is:

1. A customizable scent dispenser comprising:
an outer case;
a scent emitting element; and
an inner enclosure operable to support the scent emitting element within the outer case in a manner in which at least a portion of the scent emitting element is supported at a spacing from the outer case so as to not be in direct contact therewith, the inner enclosure having an aperture through which replenishment access to the scent emitting element can be had such that a scented composition can be imparted to the scent emitting element and a portion of the outer case delimiting a refresh access path via which access to the inner enclosure can be had for the purpose of selectively (a) replenishing the scent emitting element with a scented composition, with access to the scent emitting element occurring through the aperture of the inner enclosure: (b) removing the inner enclosure for replenishing the scent emitting element with a scent providing composition while the inner enclosure and the therein retained scent emitting element are removed from the outer case, or (c) removing the inner enclosure with the therein retained scent emitting element and substituting a new inner enclosure with its own respective scent emitting element retained therein.

2. The customizable scent dispenser as claimed in claim 1, wherein at least a portion of the outer case substantially resists the passage therethrough of liquids and vapors emanating from the scent emitting element, whereupon the outer case can be in substantially non-staining contact with an exterior surface.

3. The customizable scent dispenser claimed in claim 1, wherein the scent emitting element is formed of a fabric, a synthetic or natural sponge, a foamed plastics woven or non-woven material, or paper.

4. The customizable scent dispenser as claimed in claim 1 and further including mounting means for suspending the customizable scent dispenser from a surface.
